(19) United States
(12) Patent Application Publication
(10) Pub. No.: US 2017/0056436 A1
(43) Pub. Date: Mar. 2, 2017

(54) MOISTURE CURING ALKOXYSILANE POLYMERS

(71) Applicants: Gregory Zugates, Chelmsford, MA (US); Chetan Khatri, Acton, MA (US); Joseph Lomakin, Cambridge, MA (US); Craig Wiltsey, Waltham, MA (US)

(72) Inventors: Gregory Zugates, Chelmsford, MA (US); Chetan Khatri, Acton, MA (US); Joseph Lomakin, Cambridge, MA (US); Craig Wiltsey, Waltham, MA (US)

(21) Appl. No.: 15/254,552

(22) Filed: Sep. 1, 2016

Related U.S. Application Data
(60) Provisional application No. 62/213,497, filed on Sep. 2, 2015.

(51) Int. Cl. A61K 31/80; A61K 49/00; C08G 77/388; A61F 2/07; A61M 5/00; A61M 5/178; A61J 1/14; A61B 17/12; A61K 9/00; C08L 83/08

(52) U.S. Cl. CPC A61K 31/80; A61K 9/0019; A61K 49/0002; C08G 77/388; C08L 83/08; A61M 5/007; A61M 5/178; A61J 1/1468; A61B 17/12186; A61F 2/07

(57) ABSTRACT

The present disclosure is directed to moisture curing formulations as well as articles, devices, systems, kits and methods based on the same.

### CROSS-REFERENCE TO RELATED APPLICATION

This application claims priority to and the benefit of, U.S. Ser. No. 62/213,497, Filed Sep. 2, 2015, which is incorporated herein by reference in its entirety.

### FIELD

Moisture curing formulations as well as articles, devices, systems, kits and methods based on the same are generally described.

### BACKGROUND

Various types of moisture curable materials are known, including polysiloxane polymers having silicon-based groups provided with one or more hydrolyzable groups. These polymer crosslink upon exposure to moisture without the generation of gaseous reaction byproducts.

### SUMMARY

In various aspects of the present disclosure, injectable formulations are provided which contain a prepolymer that comprises a polymer backbone and a plurality of methylalkoxysilane groups. The methylalkoxysilane groups may, for example, comprise —Z—CH₂—SiOR¹ᵢR² groups, where R¹ is a C1-C10 hydrocarbon radical, where R² is a C1-C10 hydrocarbon radical, where Z is selected from —O—, —S—, and —NR³—, where R³ is H or a C1-C10 hydrocarbon radical, and where i=1, 2, or 3.

In certain beneficial embodiments, the prepolymer comprises a polymer backbone and a plurality of α-aminomethylalkoxysilane groups which may comprise, for example, pendant amine groups such as —NR³(—CH₂—SiOR¹ᵢR²₃₋ᵢ) groups, —N(—CH₂—SiOR¹ᵢR²₃₋ᵢ)₂ groups, —N⁺R³(—CH₂—SiOR¹ᵢR²₃₋ᵢ)₂ groups, —N⁺R³₂(—CH₂—SiOR¹ᵢR²₃₋ᵢ) groups and/or —N⁺(—CH₂—SiOR¹ᵢR²₃₋ᵢ)₃ groups, as well in-chain amine groups such as —N(—CH₂—SiOR¹ᵢR²₃₋ᵢ)— groups, —N⁺(—CH₂—SiOR¹ᵢR²₃₋ᵢ)₂— groups and/or —N⁺R³(—CH₂—SiOR¹ᵢR²₃₋ᵢ)— groups, where R¹, R², R³ and i are defined above. In certain of these embodiments, R¹=OCⱼH₂ⱼ₊₁ and R²=CₖH₂ₖ₊₁, where j may range from 1 to 10, and is typically 1 or 2, and k may range from 1 to 10, and is typically 1 or 2.

In some embodiments, which may be used in combination with any of the above aspects and embodiments, the formulations may comprise prepolymers having branched polymer backbones and/or the formulations may comprise prepolymers having linear polymer backbones.

In some embodiments, which may be used in combination with any of the above aspects and embodiments, the prepolymer may comprise a polymer backbone and a plurality of methylalkoxysilane groups that do not comprise a carbonyl group.

In some embodiments, which may be used in combination with any of the above aspects and embodiments, the prepolymer may comprise a polymer backbone selected from a polysiloxane and/or a polyalkylene oxide.

In some embodiments, which may be used in combination with any of the above aspects and embodiments, the prepolymer may comprise α-aminomethylalkoxysilane groups may be selected from α-aminomethylalkoxysilane side groups, α-aminomethylalkoxysilane terminal groups, and combinations of the same.

In some embodiments, which may be used in combination with any of the above aspects and embodiments, the α-aminomethylalkoxysilane groups may be selected from α-aminomethyltrialkoxysilane groups, α-aminomethyldialkoxyalkylsilane groups and α-aminomethylalkoxydialkylsilane groups.

In some embodiments, which may be used in combination with any of the above aspects and embodiments, the α-aminomethylalkoxysilane groups may be α-aminomethylethoxysilane groups, thereby releasing ethanol upon hydrolysis.

In some embodiments, which may be used in combination with any of the above aspects and embodiments, the α-aminomethylalkoxysilane groups may comprise pendant groups such as, for example, -L₁-NH(—CH₂—SiOR¹ᵢR²₃₋ᵢ) groups, -L₁-N(—CH₂—SiOR¹ᵢR²₃₋ᵢ)₂ groups, -L₁-N⁺H(—CH₂—SiOR¹ᵢR²₃₋ᵢ)₂ groups, -L₁-N⁺H₂(—CH₂—SiOR¹ᵢR²₃₋ᵢ) groups, and/or -L₁-N(—CH₂—SiOR¹ᵢR²₃₋ᵢ)₃ groups and/or in-chain groups such as, for example, -L₁-N(—CH₂—SiOR¹ᵢR²₃₋ᵢ)-L₂-groups, -L₁-N⁺H(—CH₂—SiOR¹ᵢR²₃₋ᵢ)-L₂- groups and/or -L₁-N⁺(—CH₂—SiOR¹ᵢR²₃₋ᵢ)₂-L₂- groups, where R¹, R², R³ and i are defined above and where L₁ and L₂ may be independently selected, for example, from a C1-C20-hydrocarbon, a polysiloxane, a poly(alkylene oxide), and a polysiloxane poly(alkylene oxide) copolymer among many other possibilities. In certain of these embodiments, R¹=OCⱼH₂ⱼ₊₁ and R²=CₖH₂ₖ₊₁, where j may range from 1 to 10, typically 1 or 2, and k may range from 1 to 10, typically, 1 or 2.

In some embodiments, which may be used in combination with any of the above aspects and embodiments, the injectable formulation may have a viscosity that is less than 2000 cp, for example, ranging from 10 cp to 2000 cp, beneficially ranging from 10 cp to 250 cp.

In some embodiments, which may be used in combination with any of the above aspects and embodiments, the injectable formulation may further comprise a solvent, carrier fluid or other diluent, to decrease the formulation viscosity and/or increase the curing rate. Beneficial examples include ethanol, isopropanol, dimethyl sulfoxide, acetone, triacetin, dimethyl isosorbide, diethylene glycol dimethyl ether, tetraethylene glycol dimethyl ether, polyethylene glycol, polypropylene glycol, propylene glycol, dipropylene glycol, glyercol, and N-methylpyrrolidone, among others.

In some embodiments, which may be used in combination with any of the above aspects and embodiments, the injectable formulation may further comprise a catalyst to alter the curing rate of the formulation. Beneficial catalysts include organic amines such as triethylenediamine, 1,8-diazabicyclo-[5.4.0]-undec-7-ene, and 1,5-diazabicyclo-[4.3.0]-non-5-ene or organometallic catalysts such as stannous octoate and zinc octoate, among others.

In some embodiments, which may be used in combination with any of the above aspects and embodiments, the injectable formulation may further comprise an imaging contrast agent. Preferred imaging agents include tantalum, tungsten, barium sulfate, zinc oxide, zinc titanate, bismuth oxide and iodinated contrast agents.

In other aspects, the present disclosure pertains to medical articles that comprise a sealed container and an injectable formulation in accordance with any of the above aspects and embodiments disposed in the sealed container.

In some embodiments, the sealed container may be selected, for example, from a capped syringe, a metal foil pouch and a glass vial.

In some embodiments, which may be used in combination with any of the above aspects and embodiments, the sealed container may further comprise an inert gas.

In other aspects, the present disclosure pertains to medical kit that comprise (a) a medical article in accordance with any of the above aspects and embodiments and (b) an insertable medical device.

In some embodiments, the medical kit may comprise one or more of the following, among others: a graft, a stent, a stent-graft, particles, a balloon, a coil, a flow diverter, a flow disruptor, a filter, a plug or other barrier.

In some embodiments, which may be used in combination with any of the above aspects and embodiments, the medical kit may comprise a catheter.

In some embodiments, which may be used in combination with any of the above aspects and embodiments, the medical kit may further include an additional injectable formulation that comprises a multifunctional species that reacts with amine groups in the prepolymer that is present within the injectable formulation.

In other aspects, the present disclosure pertains methods in which an injectable formulation in accordance with any of the above aspects and embodiments is injected into a body cavity of a patient.

In some embodiments, methods are provided for controlling flow of bodily fluid in the patient. For example, blood flow in a patient may be controlled by injecting an injectable formulation in accordance with any of the above aspects and embodiments into a blood vessel of the patient.

In some embodiments, methods for treating an aneurysm having a tissue surface are provided. The methods comprise placing a medical device having an exterior surface (e.g., a graft, a stent, a stent-graft, particles, a balloon, a coil, a plug or other barrier, etc.) within and/or in contact with at least a portion of blood vessel segment containing the aneurysm and injecting an injectable formulation in accordance with any of the above aspects and embodiments between the exterior surface of the medical device and the tissue surface of the aneurysm.

In some embodiments, which may be used in combination with any of the above aspects and embodiments, the injectable formulation is injected into the patient through a catheter.

In some embodiments, which may be used in combination with any of the above aspects and embodiments, the injectable formulation reacts in the presence of water to generate ethanol.

In some embodiments, which may be used in combination with any of the above aspects and embodiments, the injectable formulation crosslinks as a result of exposure to water that is present within the patient's body or water that is introduced to the patient along with the injectable formulation.

In some embodiments, which may be used in combination with any of the above aspects and embodiments, the injectable formulation reacts to form an implant with cured outer skin and a slow-curing interior.

In some embodiments, which may be used in combination with any of the above aspects and embodiments, an additional injectable formulation may be combined with the injectable formulation prior to or simultaneous with injection into the patient. The additional injectable formulation may comprise, for example, a multifunctional species that reacts with amine groups found in the prepolymer that is present within the injectable formulation.

The above and other aspects and embodiments as well as various advantages of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component may be labeled in every figure, nor is every component of each embodiment of the invention shown where illustration is not necessary to allow those of ordinary skill in the art to understand the invention. In the figures:

FIG. 1 shows the conventional placement of a stent-graft within an abdominal aortic aneurysm.

FIG. 2 shows an embodiment of the present disclosure in which an in-situ solidifying prepolymer formulation has been placed in the space between a stent-graft and an aneurysm sac.

FIG. 3 shows an embodiment of the present disclosure in which an injectable material in accordance with the present disclosure is delivered into an aneurysm sac via a catheter.

FIG. 4 is a model of a human aortic aneurysm that has been filled with an injectable material in accordance with the present disclosure.

FIGS. 5A-5C illustrate the filling of a model of a human cerebral aneurysm with an injectable material in accordance with the present disclosure. FIG. 5A shows the system prior to injection in which a microcatheter is placed in the unfilled aneurysm; FIG. 5B shows the system at a time when filling of the aneurysm is in progress; and FIG. 5C shows the system after the aneurysm has been filled.

### DETAILED DESCRIPTION

The present disclosure pertains to prepolymer formulations that crosslink in the presence of water and well as articles, devices, kits, systems and methods for medical treatment using the same. As used herein, “water” refers to the molecule H₂O, including isotopes of the same.

The term “polymer” is given its ordinary meaning in the art, and is used to refer to a molecule that includes a plurality of monomers. Included within the definition of “polymer” are “prepolymers,” which are a subclass of polymers that are characterized by reactive groups in the polymer chain. Such prepolymers are of particular use in the present disclosure because the reactive groups in such polymers help drive a crosslinking reaction which results in at least partial solidification of the formulations described herein. In some embodiments, a prepolymer may comprise between about 3 monomer units and about 1000 monomer units, typically between about 3 monomer units and about 150 monomer units, among other possible ranges. The polymers within the prepolymer formulation can comprise a variety of functional groups that may allow the polymers to, for example, crosslink to each other, attach to tissue or other material within the body of a subject, or interact with agents in the bloodstream of the subject (e.g., imaging agents, crosslinking agents, etc.), among other functionalities.

The term “crosslinking” is used to refer to the process whereby multiple polymer chains (i.e., two or more polymer chains) are joined together either via covalent bonding, in which case the process may be referred to as covalent crosslinking, or ionic bonding, in which case the process may be referred to as ionic crosslinking. The various crosslinking reactions described herein are covalent crosslinking reactions.

Polymers that can undergo crosslinking can comprise straight chain polymers and/or branched chain polymers (e.g., graft polymers having a main chain and a plurality of side chains, multi-arm polymers, dendritic polymers, etc.). In various cases, formulations in accordance with the present disclosure comprise prepolymers, which may contain reactive side groups (i.e., groups along the length of a polymer chain) and/or reactive terminal groups (i.e., groups at the end of a polymer chain), and crosslinking may involve reactions between side groups, reactions between terminal groups, and/or reactions between side groups and terminal groups. In some instances, the polymer formulation may be substantially free of polymers that comprise reactive side groups. In other cases, the polymer formulation may comprise a substantial amount of polymer reactive side groups. In some instances, the polymer formulation may be substantially free of polymers that comprise reactive terminal groups. In other cases, the polymer formulation may comprise a substantial amount of polymers with reactive terminal groups.

Covalent crosslinking may commence via a variety of mechanisms. In various embodiments, prepolymer formulations may crosslink once the prepolymer formulation contacts water that may be present, for example, within a patient's body or introduced from an external source (e.g., in tissue, blood, aqueous solutions, moisture in air, etc.).

In various aspects, the present disclosure pertains to prepolymers that comprise a polymer backbone and a plurality of methylalkoxysilane groups. The methylalkoxysilane groups may, for example, comprise —Z—CH₂—SiOR¹ᵢR²₃₋ᵢ groups, where R¹ is a C1-C10 hydrocarbon radical, where R² is a C1-C10 hydrocarbon radical, where Z is selected from —O—, —S—, and —NR³—, where R³ is H or a C1-C10 hydrocarbon radical, and i=1, 2, or 3. In certain beneficial embodiments, the prepolymers comprise a polymer backbone and a plurality of α-aminomethylalkoxysilane groups, examples of which include prepolymers that comprise, for example, —NR³(—CH₂—SiOR¹ᵢR²₃₋ᵢ) groups, —N(—CH₂—SiOR¹ᵢR²₃₋ᵢ)₂ groups, —N⁺R³(—CH₂—SiOR¹ᵢR²₃₋ᵢ)₂ groups, —N⁺R³₂(—CH₂—SiOR¹ᵢR²₃₋ᵢ) groups, —N⁺(—CH₂—SiOR¹ᵢR²₃₋ᵢ)₃ groups, —N(—CH₂—SiOR¹ᵢR²₃₋ᵢ)— groups, —N⁺(—CH₂—SiOR¹ᵢR²₃₋ᵢ)₂— groups and/or —N⁺R³(—CH₂—SiOR¹ᵢR²₃₋ᵢ)— groups, where R¹ is a C1-C10 hydrocarbon radical, R² is a C1-C10 hydrocarbon radical, R³ is H or a C1-C10 hydrocarbon radical, and i=1, 2, or 3. In certain of these embodiments, R¹=OCⱼH₂ⱼ₊₁ and R²=CₖH₂ₖ₊₁, where j may range from 1 to 10, typically 1 or 2, and k may range from 1 to 10, typically, 1 or 2. In certain preferred embodiments, j=2 and k=1.

Thus, while prepolymers comprising α-aminomethylalkoxysilane groups are typically described herein, it should be understood that the present disclosure is not so limited.

In various embodiments, the disclosure is directed to prepolymers that comprise α-aminomethylalkoxysilane groups (e.g., α-aminomethyltrialkoxysilane groups, α-aminomethyldialkoxyalkylsilane groups and/or α-aminomethylalkoxydialkylsilane groups), which are capable of rapidly crosslinking in the presence of water. The α-aminomethylalkoxysilane groups contain an electron donating nitrogen close to the alkoxysilane group, which greatly increases the reactivity of such groups relative to counterparts that lack an amine at the alpha position relative to the alkoxysilane (e.g., aminopropylalkoxysilanes), resulting in near instant crosslinking of these materials upon exposure to water.

Liquid formulations based on prepolymers comprising α-aminomethylalkoxysilane groups are described which, when delivered into a water-containing environment, immediately increase in viscosity while also having the ability to conform to a target site (e.g., a body cavity such as an aneurysm) into which the formulations are delivered. Prepolymers comprising α-aminomethylalkoxysilane groups undergo reaction with water to form a solid, with the release of the corresponding alcohol associated with the alkoxy group. In certain beneficial embodiments, prepolymers comprising α-aminomethylethoxysilane groups are employed, in which case the corresponding alcohol associated with the alkoxy group that is released is ethanol.

In some embodiments, upon exposure to water, such formulations may have a fast forming outer “skin” and a slower hardening interior that permits retention at the target site, even under high flow conditions, while maintaining material flow and/or flexibility to fill complex geometries. The interior of the material cures more slowly due to the time for water penetration and diffusion. This skinning effect is very rapid (e.g., typically occurring within a few seconds upon contact with an aqueous solution) and provides material cohesion and resistance to deformation that could lead to undesirable flow outside the targeted area (e.g., outside an aneurysm sac, which could lead to embolism). During the course of delivery, the skin of the material may split releasing some uncured prepolymer formulation from the interior. The uncured liquid, however, will quickly react and cure, forming a new “skin”.

Reaction of α-aminomethylalkoxysilane groups in the presence of water results in Si—O—Si bond formation, which has been reported to be due to hydrolysis of alkoxysilanes to form hydroxysilanes, which subsequently condense to form S—O—Si bonds.

Such groups may be provided, for example, by reacting a polymer having amine side groups and/or amine terminal groups with a halomethylalkoxysilane (e.g., a halomethyltrialkoxysilane or halomethyldialkoxyalkylsilane or halomethylalkoxydialkylsilane. For example, the halomethylalkoxysilane may be of the formula X—CH₂—SiOR¹ᵢR²₃₋ᵢ, more typically, X—CH₂—Si(OCⱼH₂ⱼ₊₁)ᵢ(CₖH₂ₖ₊₁)₃₋ᵢ, where R¹, R², j, k, and i are defined above and X is a halogen (i.e., F, Cl, Br, I, etc.), typically Cl. The reaction may be facilitated by high temperature (e.g., 90° C., among other values), long times (e.g., 24 hours, among other values), and/or the addition of a base catalyst such as triethylamine.

Conjugation of each of these agents to an amine (e.g., a terminal-group amine and/or a side-group amine, on a polymer chain) provides 1, 2 or 3 alkoxy functional groups per amine, which can be used to alter the final crosslinked density and mechanical properties of the cured material. For example, more alkoxy functional groups and/or lower prepolymer molecular weight may result in crosslinked materials with higher moduli and lower elongations at break, while less alkoxy functional groups and/or higher prepolymer molecular weight may result in crosslinked materials with smaller Moduli and higher elongations to break.

In some embodiments, the polymer to be reacted with the halomethylalkoxysilane may comprise primary amine groups (e.g., -L-NH₂ groups, including side and/or terminal groups), secondary amine groups (e.g., -L²-NH-L²- and/or -L²-NHL² groups, including side and/or terminal groups), and/or tertiary amine groups (e.g., -L²-N⁺(L²)-L³-groups and/or -L²-N⁺L²L³ groups, including side and/or terminal groups), where L, L¹, L² and L³ may independently be, for example, a bond or an organic radical, for example, C1-C20 hydrocarbon (e.g., C1-C20 alkyl), or a polymer linking group, for instance, a polysiloxane, a poly(alkylene oxide) linking group such as poly(ethylene oxide), poly(propylene oxide) or poly(tetramethlyene oxide), among other possibilities.

One specific reaction between (a) a poly(dialkylsiloxane-co-aminoalkylalkylsiloxane), specifically, a poly(diC1-C10-alkylsiloxane-co-aminoC1-C20-alkyl-C1-C10-alkylsiloxane), more specifically, a poly(dimethylsiloxane-co-aminoC1-C20-alkyl-methylsiloxane), in particular, a poly(dimethylsiloxane-co-aminopropylmethylsiloxane) and (b) a halomethyltriC1-C5-alkoxysilane, halomethylC1-C5-alkyldiC1-C5-alkoxysilane or halomethyldiC1-C5-alkylC1-C5-alkoxysilane, specifically, chloromethylmethyldiethoxysilane (CMMDES), in the presence of base (i.e., triethylamine base) and solvent (i.e., toluene) is shown here:

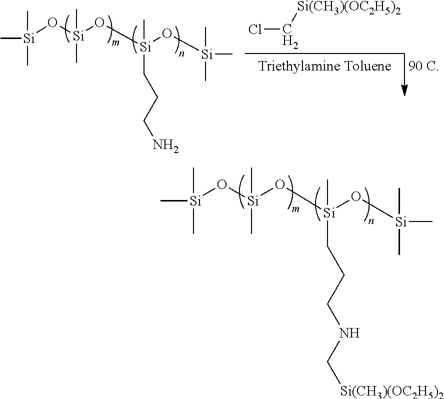

where n and m are integers.

Although reaction of a single halomethylalkoxysilane is shown, resulting in the formation of a secondary amine, in various embodiments, the halomethylalkoxysilane may be over-indexed relative to the amine such that multiple reactions occur at each amine, thereby increasing the functionality of the material. In this way, one may form a secondary amine (one reaction per amine), tertiary amine (two reactions per amine) and/or quaternary amine (three reactions per amine). For reaction between a given amine and a triethoxy chloromethyl compound, this would result in 3, 6, and 9 triethoxy groups per amine, respectively.

After reaction between a polymer having amine side and/or terminal groups and a halomethylalkoxysilane, the α-aminomethylalkoxysilane prepolymer product can be purified by filtering any precipitate that may be formed, for example, a precipitated reaction product of the base and the hydrogen halide that is produced by the reaction (e.g., a hydrohalide salt of the base, which is triethylamine hydrochloride salt in the preceding reaction scheme). The solvent (e.g., toluene in the preceding reaction scheme) and any residual base catalyst (e.g., triethylamine in the preceding reaction scheme) and/or halomethylalkoxysilane reactant (e.g., CMMDES in the preceding reaction scheme) may then be removed from the reaction product, for example, by distillation or another suitable technique.

Using these and other techniques, a variety of prepolymers comprising α-aminomethylalkoxysilane groups may formed, examples of which include prepolymers which have side groups and/or end groups of the formula: -L-NH(—CH₂—SiOR¹ᵢR²₃₋ᵢ), -L-N(—CH₂—SiOR¹ᵢR²₃₋ᵢ)₂ and/or -L-N⁺(—CH₂—SiOR¹ᵢR²₃₋ᵢ)₃, among other possibilities, for instance, -L-NH(—CH₂—Si(OCⱼH₂ⱼ₊₁)ᵢ(CₖH₂ₖ₊₁)₃₋ᵢ), -L-N(—CH₂—Si(OCⱼH₂ⱼ₊₁)ᵢ(CₖH₂ₖ₊₁)₃₋ᵢ)₂ and/or -L-N⁺(—CH₂—Si(OCⱼH₂ⱼ₊₁)ᵢ(CₖH₂ₖ₊₁)₃₋ᵢ)₃, where L, R¹, R², j, k, and i are defined above. In the particular scheme shown above, j=2, k=1, i=2, and L=C3-alkyl, specifically, n-propyl.

Another example of a polymer that may be produced is as follows:

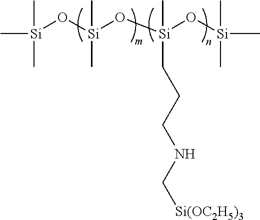

where n and m are integers, j=2, i=3 and L=C3-alkyl, specifically, n-propyl. As above, although reaction of a single halomethylalkoxysilane is shown, resulting in the formation of a secondary amine, in various embodiments, the halomethylalkoxysilane may be over-indexed relative to the amine such that multiple reactions occur at each amine, thereby forming tertiary and/or quarternary amine products.

While the preceding example employs a prepolymer comprising —NH₂ side groups, as previously noted, in some embodiments, prepolymers can be employed that comprise —NH₂ terminal groups, for example, -L-NH₂ terminal groups, where L is defined above. A particular example of such a prepolymer is an aminoalkyl-terminated polydialkylsiloxane, more particularly, an aminoC1-C20-alkyl-terminated polydiC1-C10-alkylsiloxane, even more particularly, an aminoC1-C20-alkyl-terminated polydimethylsiloxane, specifically, an aminopropyl-terminated polydimethylsiloxane, which when reacted with a halomethyltrialkoxysilane, a halomethyldialkoxyalkylsilane and/or a halomethylalkoxydialkylsilane, more specifically, a halomethyltriethoxysilane, yields the following prepolymer:

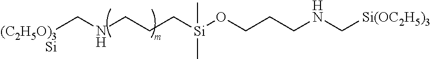

where m is an integer. As elsewhere herein, although reaction of a single halomethylalkoxysilane is shown, resulting in the formation of a secondary amine, in various embodiments, the halomethylalkoxysilane may be over-indexed relative to the amine such that multiple reactions occur at each amine, thereby forming tertiary and/or quarternary amine products.

In some embodiments, a significant portion of the primary amine functional groups in the polymer (e.g., ranging from 20% to 90%) are not reacted with the halomethylalkoxysilane, for example, yielding a prepolymer product comprising a number of -L-NH₂ side groups and/or -L-NH₂ terminal groups, where L is defined above. This result can be achieved by underindexing the halomethylalkoxysilane relative to the primary amine functional groups on the polymer such that partial conversion of the functional groups occurs. The resulting prepolymer may can be used to generate cured materials with smaller Moduli and higher elongations to break in some embodiments. One specific embodiment of such a prepolymer is shown here:

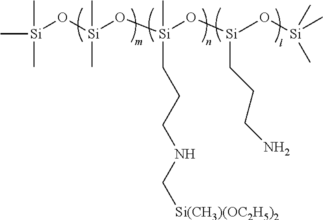

where l, m and n are integers.

While polysiloxane polymers are exemplified in the foregoing embodiments for functionalization with α-aminomethylalkoxysilane groups, a wide variety of additional polymers may be likewise functionalized, including, for example, polyethers including polyalkylene oxides (e.g., polyethylene oxide, polybutylene oxide, polyethylene oxide-polybutylene oxide copolymers, polytetramethylene oxide, etc.), polypolyolefins (e.g. polyethylene, polypropylene, ethylene vinyl acetate copolymer, etc.), polystyrenes (e.g., polystyrene, acrylonitrile-butadiene-styrene copolymers, styrene-acrylonitrile copolymers, acrylonitrile-styrene-acrylate copolymers, methacrylate-acrylonitrile-butadiene-styrene copolymers, styrene-butadiene copolymers, etc.), polyesters (e.g., polyethylene terephthalate, polybutylene terephthalate, etc.), polycarbonates, phenolic polymers, polyvinyl chloride, polysulfones including polyethersulfone, poly(meth)acrylates, polyetheretherketones, thermoplastic elastomers (TPE) (e.g., polyamide TPE, copolyester TPE, olefinic TPE, styrenic TPE, urethane TPE, etc.), fluoropolymers (e.g., polytetrafluoroethylene, fluorinated ethylene propylene (FEP) copolymer, ethylene chlorotrifluoroethylene copolymers, ethylene tetrafluoroethylene copolymers, polyvinyl fluoride, polyvinylidene difluoride, etc.), polyallylamine, polyethylenimine, diethylaminoethyl dextran, dimethylaminoethyl methacrylate, polyamidoamines, and other aminated or hydroxylated polymers, among many others.

Such polymers may be may be commercially available in forms having primary amine functionality, for example, having terminal —NH₂ groups. For example, JEFFAMINE polytheramines, available from Huntsman Corporation, Salt Lake City, Utah, USA, contain primary amine groups attached to the ends of polyethers (e.g., polyethylene oxide, polybutylene oxide, poly(ethylene oxide-co-butylene oxide) copolymers, and polytetramethylene oxide).

Where polymers having primary amine functionality are not commercially available, various strategies are known in the art for introducing amine functionality to such polymers. For example, hydroxyl-functionalized polymers, which are often more commonly available than amine-functionalized polymers, may be used to create reactive alkoxysilane polymers. Synthesis of reactive alkoxysilane polymers using such polymers may require a stronger base to facilitate the reaction (e.g., sodium hydride). In other embodiments, hydroxyl groups may be converted to primary amine groups, for example, using methods known in art.

The above and other synthesis schemes may be used to generate highly reactive, moisture sensitive, α-aminomethylalkoxysilane functionalized prepolymers of varied backbone composition, architecture (e.g., linear or branched) and molecular weight.

Typical prepolymer molecular weights may range, for example, from 500 to 100,000 Daltons, more typically from 1,000 to 10,000 Daltons. Where prepolymers having polysiloxane backbones are synthesized, molecular weights ranging from 1,000 to 10,000 Daltons are particularly preferred, among other possibilities

The α-aminomethylalkoxysilane functionalized prepolymers described herein may be used to formulate of low viscosity, fast reacting injectable formulations. In this regard, low viscosity formulations are desirable in that the formulations may be delivered via a small diameter tube (e.g., a catheter ranging from 3F to 18F, more beneficially, ranging from 3F to 7F) without the need for excessive amounts of pressure to achieve a suitable flow rate. Beneficially, formulations described herein have viscosities less than 2000 cp (e.g., ranging from 10-2000 cp), beneficially less than 1000 cp (e.g., ranging from 10-1000 cp), more beneficially less than 500 cp (e.g., ranging from 10-500 cp), even more beneficially less than 250 cp (e.g., ranging from 10-250 cp). Viscosity may be measured using a rheometer or rotational viscometer.

Formulations in accordance with the present disclosure may also comprise crosslinkable groups in addition to α-aminomethylalkoxysilane crosslinking groups.

In this regard, as previously noted, various formulations described herein have a rapidly forming outer “skin” and a slower hardening interior. The interior of the material cures more slowly due to the time required for transport of water to the interior. In certain embodiments, a secondary crosslinking mechanism, which proceeds more slowly than the primary α-aminomethylalkoxysilane crosslinking mechanism, may be employed, for example, to enhance crosslinking in the interior.

Secondary crosslinking may be achieved in the formulations of the present disclosure by providing suitable paired reactive groups. Examples of reactive groups on polymer chains that can be paired to produce crosslinking include, for example, amines and acrylates, amines and methacrylates, amines and epoxides, amines and isocyanates, hydroxyls and isocyanates, amines and NHS-esters, thiols and maleimides, azides and alkynes (i.e. “click chemistry”), and acid chlorides and alcohols.

It may be desirable, in some embodiments, to keep these paired chemicals separate until they are introduced into the implant site to prevent unwanted crosslinking outside the implant site. For example, a secondary formulation containing one of the pair of chemicals may be introduced to the implant site from a container separate from the container used to introduce the prepolymer formulation, which contains the other of the pair of chemicals. Upon combining the primary and secondary formulations, crosslinking occurs, and viscosity may be increased. In some cases, the crosslinking proceeds until a solid material (e.g., a solid elastomeric implant) is formed.

The prepolymers described herein comprise secondary amine groups associated with the α-aminomethylalkoxysilane groups as indicated above, and in various embodiments, may primary amine groups as well (e.g., where only partial conversion of primary amine functional groups in the polymer is carried out). Crosslinking based on these amine groups may be carried out by forming a secondary formulation that contains multifunctional species that are reactive with secondary and/or primary amines such as, for example, multifunctional acrylate species, multifunctional methacrylate species, or multifunctional isocyanate species, among other possibilities.

In certain embodiments where secondary crosslinking is desired, crosslinking based on reaction of amines and multi-functional acrylates, or amines and multi-functional methacrylates, may be employed. In these embodiments, secondary formulations containing such species may be combined with formulations containing α-aminomethylalkoxysilane functionalized prepolymers, thereby crosslinking the prepolymers via the amine groups contained in the prepolymers.

Examples of multifunctional acrylates for use in such secondary formulations include reaction products of acrylic acid with species containing multiple hydroxyl groups, for example, diols, triols, tetraols, pentols, hexols and hydroxyl terminated polymers. Specific examples include α,ω-C2-C20-alkane diol acrylates such as 1,2-ethane diacrylate, 1,3-propane diacrylate, 1,4-butane diacrylate, 1,5-hexane diacrylate, 1,6-hexane diacrylate, and so forth, ethylene glycol diacrylate, diethylene glycol diacrylate, glycerol diacrylate, glycerol triacrylate, pentaerythritol diacrylate, pentaerythritol triacrylate, pentaerythritol tetraacrylate, polysiloxane diacrylates, polyethylene glycol diacrylates, polypropylene glycol diacrylates, and (acryloxypropyl)methylsiloxane-dimethylsiloxane copolymers. Examples of multifunctional methacrylates for use in such secondary formulations include versions of the foregoing species in which methacrylates are substituted for acrylates.

Formulations, articles, systems, kits and methods are described herein which are useful in medical treatment procedures in which polymer implants are formed in-vivo in a patient. As will be recognized by those skilled in the art, although the present disclosure is described with specific reference to the use of prepolymer formulations for forming implants within aneurysm sacs, the prepolymer formulations of the present disclosure may be used in other medical procedures, including the formation of implants in various body cavities such as abdominal, pelvic, and cardio thoracic cavities and other lumen structures such as arteries, veins, fallopian tubes, and the stomach (e.g., for obesity treatment), among others, and may be used to form implants that are in contact with, for example, normal tissue, distended tissue, diseased tissue, injured tissue, internal organs, etc. As used herein, “aneurysm sac” refers to the sac formed by the localized dilation in a blood vessel at an aneurysm site.

In various embodiments, the polymer implants of the present disclosure are formed “in-situ.” That is, the implants are formed by the reaction of prepolymer(s) in a water-containing environment simultaneously with, or shortly after, delivery to an implant site. In this manner, the present disclosure provides for the in-vivo formation of polymer implants. This is in contrast to pre-formed implants, which are formed prior to the time that they are delivered into the body. Such in-situ solidifying implants preferably fill the implant site volume, resulting in conformal contact with the body cavity walls, medical devices and, in some embodiments, partial penetration into blood vessels and other lumens.

The polymer implants of the present disclosure may possess attributes that make them particularly suitable for use within the body. For example, implants are described herein which are biocompatible. In some instances, the polymer implants may be sufficiently elastic to allow for body movement while being sufficiently stiff to support body tissues. In some embodiments, the formulation may be adjusted so that it wets tissues effectively. The prepolymer formulations and/or implants formed therefrom may also contain imaging contrast agents, allowing for the visualization of the implant. These and other aspects of the implants used in the present disclosure are more fully described herein.

In some embodiments, the prepolymer formulations described herein may comprise fluid prepolymers in the substantial absence of a solvent, carrier fluid or other diluent. In other instances, the prepolymers in the formulations may be suspended in a carrier fluid or other diluent or dissolved in a solvent to create a homogeneous phase.

Beneficial solvents, carrier fluids or other diluents for use in the present disclosure may be selected from one or more of the following: ethanol, dimethylsulfoxide (DMSO), isopropanol, toluene, diglyme, polyethyleneoxide, polyethylene glycol dimethyl ether, propylene carbonate, propylene glycol, dipropylene glycol, polypropylene glycol, polyethylene glycol, glycerol, acetone, dimethyl isosorbide, triacetin, N-methylpyrrolidone, triethyl citrate, diethylene glycol dimethyl ether, tetraethylene glycol dimethyl ether, dimethyl esters of diacids (e.g., diethyl malonate, dimethyl adipate), and oils (vegetable, olive, castor, etc.), among many other possibilities.

In various embodiments, the implants described in the present disclosure are polysiloxane implants formed in-situ from a one-part formulation comprising an α-aminomethylalkoxysilane functionalized polysiloxane prepolymer. The polysiloxane is preferably a polydimethylsiloxane in some embodiments.

Optionally, the prepolymer formulations described herein may also contain multiple polymer species, diluents, catalysts, surfactants, chain extenders, crosslinkers, pore openers, fillers, and/or plasticizers. The prepolymer formulations may optionally include a coagulant such as thrombin, kaolin, glass, chitosan, or other hemostatic agent. The prepolymer formulations may optionally include a visualization material such as an imaging contrast agent, for instance, a radiopaque agent that renders the resultant implant visible through fluoroscopy or other visualization techniques.

The properties of the prepolymer formulations used to form the polymer implants may be tailored to achieve a desired result. For example, in some embodiments, the viscosity of the prepolymer formulation is tailored such that the prepolymer formulation is better able to permeate the implant site and create conformal contact with the tissue wall and/or medical device placed within the body cavity. An overly viscous prepolymer formulation may require excessive pressure to deploy within the implant site. In addition, an overly viscous prepolymer formulation may inhibit the polymer from accessing interstitial spaces. One of ordinary skill in the art will be able to produce the desired viscosity for a given polymer type by, for example, adjusting the molecular weight of the prepolymer or by the addition of one or more suitable solvents, carrier fluids or other diluents such as those listed above, among others.

In some embodiments, properties or composition of the prepolymer formulation may be chosen to achieve a desired hydrophilicity or hydrophobicity. The hydrophilicity of the prepolymer formulation may be selected, in some instances, such that the surfaces (e.g., tissue surfaces) within an implant site are appropriately wetted. Generally, a material with increased hydrophilicity will have a greater tendency to wet soft tissues surfaces and to react more quickly because of better mixing with blood. However, the prepolymer formulation and resulting polymer implant may be, in some cases, somewhat hydrophobic such that they do not dissolve into biological fluids. In various beneficial embodiments, hydrophilic prepolymer formulations are provided which are capable of conformally wetting interior surfaces of an implant site while remaining contained within the cavity. In some embodiments, the composition of the prepolymer formulation may be selected to achieve a desired hydrophilicity. For example, in some embodiments, the monomer forming the prepolymer can be varied to change hydrophilicity.

In some embodiments, the polymer implants described herein may have favorable mechanical properties. In some embodiments, the polymer implants are elastomeric. The term “elastomer” as used herein, refers to a polymer that can return to the approximate shape from which it has been substantially distorted by an applied stress. In some cases, the elastomeric polymer implants described herein may comprise a polymer having a bulk modulus of between about 0.05 MPa and about 10 MPa: 0.05 MPa and about 100 MPa; and 0.05 MPa and about 500 MPa. Elastomeric polymers may be particularly suitable for use in making polymer implants because they are capable sustaining stress without permanently deforming, while providing adequate support for body organs and tissues.

The time required to form the polymer implant after exposure of the formulation to the implant site and the final mechanical and physicochemical properties of the polymer implant can depend on such factors as the composition of the prepolymer formulation and its hydrophobicity, the density of pendant groups (e.g., crosslinking groups), relative positions of the pendant groups (e.g., crosslinking groups), the composition of any secondary crosslinking formulations and other factors.

The polymer implants described herein may be used, in some embodiments, to prevent or limit the movement of a bodily fluid within the implant site, relative to an amount of movement of bodily fluid that would occur under essentially identical conditions in the absence of the polymer implant. “Essentially identical conditions,” in this context, means conditions that are similar or identical other than the presence of the polymer implant. For example, otherwise identical conditions may mean that the implant site is identical, the conditions within the cavity are identical, but where no polymer implant is located within the implant site. In some embodiments, the polymer implant may be used to reduce the movement of blood or other bodily fluid within an implant site.

The movement of bodily fluids may be prevented or limited over a relatively long period of time. In the one embodiment, the implant forms a permanent hemostatic implant within the implant site.

In some cases, the movement of bodily fluids may be prevented or limited due to a physical seal created between an aneurysm wall or collateral vessel walls (e.g. inferior mesenteric artery, lumbar arteries), another medical device (e.g., stent, graft, stent graft, balloon, particles, embolization coil, flow diverter, flow disruptor, embolization plug, embolic filter, or other barrier) and the surface of the implant. This seal may be due to chemical bonding between the tissue surface and implant and/or the highly conformal contact of the implant with the tissue surfaces combined with the implant's tendency to induce coagulation of blood. In some embodiments, the polymer implant may be covalently bonded to a surface within the implant site, for example, through a pendant group on the prepolymer. In addition, the implant may partially penetrate collateral vessels within the implant site to further limit blood flow into the sac.

In some instances, an active agent may be delivered to the implant site with the prepolymer. In some embodiments, the prepolymer formulation may comprise an active agent. For example, an active agent (or a plurality of particles containing one or more active agents) may be dispersed within the prepolymer formulation or an active agent may be included as pendant groups on the prepolymer. Example of such active agents include, but are not limited to, antifibrinolytic compounds (e.g., aminocaproic acid, tranexamic acid, etc.), anti-fibrotic compounds, antimicrobial compounds (e.g., antibiotics), anti-inflammatory compounds, analgesics, pro-coagulant compounds, statins, growth factors, agents that stimulate desirable cellular responses such as fibroplasia, angiogenesis and epithelialization, and vasoconstrictors. Active agents that comprise groups that are reactive with the α-aminomethylalkoxysilane functionalized prepolymer may, in some cases, be isolated from the same within the prepolymer formulation, for example, to prevent unwanted reaction during the crosslinking step. Isolation can be achieved by encapsulating active agents into secondary particles and loading them into the prepolymer formulation at the time of delivery. In addition, encapsulation may be used to release the active agents at a controlled rate.

The prepolymer formulation described herein may be combined with a second agent (and, optionally, a third agent, fourth agent, etc.), in some cases, before or after the prepolymer formulation is transported to the implant site. The second agent may comprise, for example, a compound (e.g., water, catalyst, etc.) that leads to or accelerates the rate of crosslinking. The second agent may comprise, for example, a compound that leads to crosslinking that would not have occurred in the absence of the second agent. For example, in some embodiments, the second agent may comprise a multifunctional (meth)acrylate compound as noted above. In some cases, the second agent can be native in the body (e.g., blood). In other cases, the second agent may originate from outside the body. For example, the second agent may be, for example, supplied to the implant site in a secondary formulation, along with the prepolymer formulation.

In some embodiments, the combination of a second agent with the prepolymer formulation produces a polymer implant with significantly different mechanical properties (e.g., elastic modulus, yield strength, breaking strength, etc.) than would have been produced in the absence of the second agent. For example, addition of the second agent may lead to increased crosslinking among polymer molecules, potentially producing a stiffer implant. In another embodiment, the second agent may have a high molecular weight, such that the distance between crosslinks is high, and the resulting implant is softer.

The combination of the second agent with the prepolymer formulation may, in some embodiments, prevent or limit the flow of blood into the implant site, relative to an amount of blood flow that would occur under essentially identical conditions in the absence of the second agent. In some embodiments, blood flow may be reduced due to the increased rate of crosslinking or implanting mentioned above. In some cases, the second agent may comprise a pro-coagulant compound (e.g., thrombin, fibrinogen, factor X, factor VII, kaolin, glass, chitosan, or other hemostatic agent).

The second agent may be provided in a secondary formulation stored in a container separate from the prepolymer formulation, for example, to prevent unwanted reaction between the prepolymer formulation and the second agent outside the implant site. In some embodiments, a container can be used that keeps the prepolymer formulation and a secondary formulation comprising the second agent separated while stored or transported, but allow for mixing at the outlet nozzle or within the implant site when the contents are expelled. The outlet nozzle can mix multiple components (>2) in a static or dynamic manner. Examples of static mixers are helical mixers, Low Pressure Drop (LPD) mixers, square element mixer (Quadro), GXF and Interfacial Surface Generator (ISG) mixers. Examples of dynamic mixers are impellers, and rotary static mixers. Nozzles may handle low and high pressure differentials during dispensing. The container may also be designed to mix the components immediately prior to dispensing by breaking the barrier between each of the components and allowing them to mix. Mixing can occur manually such as shaking the canister or chambers can be under vacuum and when the barrier is broken a vortex will be created to mix the components.

In some embodiments, the implant can be imaged. The ability to image the implant can allow for efficient localization and repair of an injury, stabilization of a wound, etc. In some embodiments, pendant groups on the prepolymer or polymer implant can be utilized to aid in imaging the implant site. For example, a contrast agent can be introduced into the blood stream of a subject in which the implant site is located, and the contrast agent may be capable of selectively binding to pendant groups of the polymer. Examples of contrast agents include, for example, colored, fluorescent, or radiopaque imaging entities. Examples of radiopaque imaging entities include, for example, barium-based substances, iodine-based substances, tantalum powder, tantalum oxide powder, tantalum-based substances, and zirconium dioxide. In another embodiment the implant itself provides sufficient radio-contrast to surrounding tissues to facilitate visualization. In some embodiments, the contrast agents emit electromagnetic radiation in the near-infrared range (e.g., about 700 to about 1000 nm) upon interacting with the polymer implant. As a specific example, quantum dots (QD) may be used as contrast agents. In some cases, fluorescent organic tags (e.g. fluoroscein isocyanate) or radio-opaque chelating groups (e.g., Gd3+) can be used with appropriate imaging equipment. In another example, the contrast agents listed above may be attached as pendant groups to the polymer or dispersed in the prepolymer formulation to aid in visualization. In some embodiments, tantalum, titanium, gas bubbles or barium sulfate powder may be physically mixed with the prepolymer formulation for visualization. To provide a time-dependent contrast, the implant may include bio-erodible particles or fibers which include the contrast agent. Following exposure to a physiological environment, the particles or fibers will erode and release the contrast agent which can then be eliminated from the implant site. This can provide implants which become less radio-opaque, for example, over time post-delivery. This may be advantageous to users who want to evaluate location of the implant for some time after implantation, but then do not desire to have a radio-opaque implant providing imaging artifacts which limit assessment of surrounding tissues. In certain embodiments, the radio-opacity will decrease substantially within three months of implantation.

In another embodiment drug-loaded entities are incorporated in prepolymer formulation at or before administration. Incorporation of drug-loaded entities into a prepolymer formulation during administration is accomplished by those methods known to those skilled in the medical and pharmaceutical formulation arts. Examples of drug-loaded entities include: microspheres, microfibers, core-sheath microfibers, core-sheath nanofibers, nanoparticles, nanospheres, nanofibers or pure particles of drug.

Because the α-aminomethylalkoxysilane functionalized prepolymers described herein are reactive with water, including water in the surrounding atmosphere, they are typically contained within sealed containers. Preferred containers include, for example, capped syringes, glass vials, metal foil pouches and ampules. In some embodiments, any space not occupied by the prepolymer formulation will be occupied by a vacuum or an inert gas (e.g., nitrogen, helium, argon, etc.). In some embodiments, the sealed containers will be packaged along with a desiccant.

In some embodiments, a kit including one or more of the formulations previously discussed (e.g., an injectable prepolymer formulation, a device comprising such a prepolymer formulation, an injectable secondary formulation comprising a second agent, a device comprising such a secondary formulation, etc.) and a delivery system that can be used to deliver the formulations previously discussed and create a polymer implant is described. A “kit,” as used herein, typically defines a package or an assembly including one or more of the formulations previously described as well as other compositions or components associated with the invention. In certain cases, some of the compositions may be constitutable or otherwise processable, for example, by the addition of a suitable solvent, other species, or source of energy (e.g., UV radiation), which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, fits, filters, rings, clamps, wraps, patches, containers, tapes, adhesives, and the like, for example, for using, administering, modifying, assembling, storing, packaging, preparing, mixing, diluting, and/or preserving the components for a particular use.

A kit of the present disclosure may, in certain cases, include different compositions that can be mixed to form a product. In certain embodiments, the kit may include physically separated chambers to hold a first formulation comprising an α-aminomethylalkoxysilane functionalized prepolymer and at least one second formulation comprising at least one second agent (e.g., such that at physician or other user may select what properties are needed and then select from one or more second formulations), and a mechanism that is activated by a user or a machine for discharging the formulations and/or mixing them together. As a non-limiting example, the kit may include a dual barrel syringe having first and second chambers that contain the first and second formulations, wherein the first and second chambers are physically separated, for example by a wall. In this example, the user may depress the plunger of the dual-barrel syringe to eject the first and second formulations from the first and second chambers. In certain embodiments, the kit also includes a static mixing nozzle, a dynamic mixing nozzle, an impeller, or a mixing chamber to permit the first and second formulations to mix prior to or during discharge. In some embodiments, the kit includes a container or chamber within a delivery device that contains, or is configured to contain, saline or another fluid intended to cause the solidifying reaction of the prepolymer formulations delivered in accordance with the present disclosure.

A kit of the present disclosure may, in some cases, include instructions in any form that are provided in connection with the compositions of the present disclosure in such a manner that one of ordinary skill in the art would recognize that the instructions are to be associated with the compositions of the present disclosure. For instance, the instructions may include instructions for the use, modification, mixing, diluting, preserving, administering, assembly, storage, packaging, and/or preparation of the formulations and/or other compositions or components associated with the kit. In some cases, the instructions may also include instructions for the delivery and/or administration of the formulations, for example, for a particular use, e.g., to a sample and/or a subject, or to deliver the formulations of the present disclosure into contact with bodily tissues to prevent, limit, or otherwise control bleeding or the flow of other bodily fluids. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Methods of using the prepolymer formulations of the present disclosure for the treatment of aneurysms are now provided.

One specific clinical application in which present disclosure can be used is in the treatment of aneurysms. Generally, an aneurysm is an abnormal widening or ballooning of a portion of a blood vessel due to weakness in the vessel wall. If left untreated, aneurysms can grow large and rupture, causing internal bleeding which is often fatal. Two locations in which aneurysms are commonly found are in the abdominal aorta and the brain.

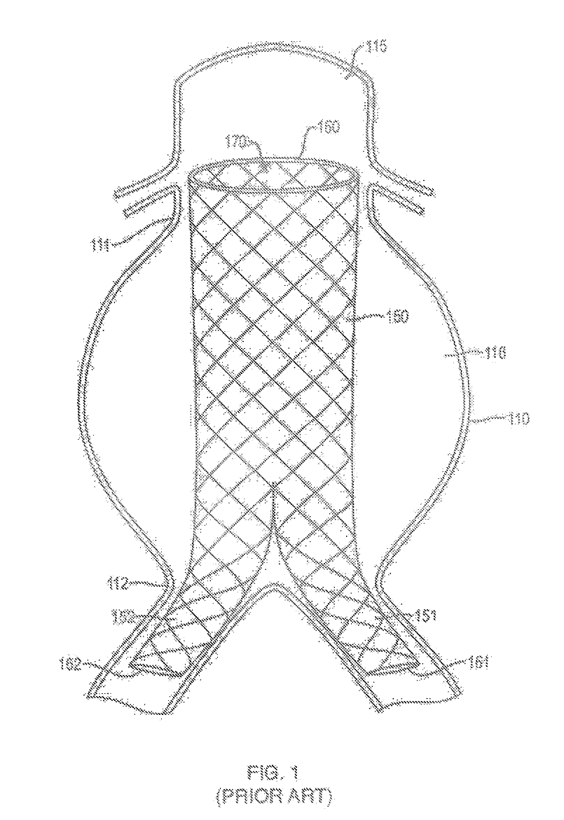

Abdominal aortic aneurysms (“AAAs”) are conventionally treated by surgical removal or by endovascular repair. If the AAA is surgically repaired, a major incision is made in the abdomen or chest to access and remove and/or repair the aneurysm, and the aneurysmal segment of aorta is replaced or supplemented with a tubular graft of synthetic material such as Dacron® or Teflon®. If instead it is treated by endovascular aneurysm repair (“EVAR”), the AAA is accessed via catheter using minimally invasive techniques rather than through an open surgical incision. A graft or stent-graft is delivered through the catheter and is forced to expand or self-expands as it is expelled from the catheter to bridge the aneurysm to form a stable channel for blood flow. FIG. 1 shows an aneurysm 110 in an abdominal aorta 115 after treatment by the placement of a stent-graft 150, as is known in the art. With the increased use of EVAR in recent years, a higher incidence of endoleaks has been observed. An endoleak results from blood that is still able to access the aneurysm sac 116 after placement of the graft or stent-graft Such a leak could be caused by an insufficient seal at the ends of the graft (referred to as a “type I” leak), retrograde flow into the aneurysm from collateral vessels (a “type II” leak), a defect in the graft (a “type III” leak), and flow through any porosity in the graft (a “type IV” leak). Such endoleaks represent a significant possible drawback to EVAR procedures as they may lead to aneurysm expansion or rupture. Endoleaks are less of a concern following surgical repair of AAA, but the surgical procedure is significantly more invasive and has higher mortality and morbidity.

The present disclosure provides formulations, articles, compositions, devices, systems, kits and methods improved an improved EVAR device and system which address endoleaks would provide a significant improvement in patient care.

Although the present disclosure is described with specific reference to the treatment of AAAs, it should be appreciated that it is applicable to the treatment of other conditions, including other aneurysms, such as those in the descending thoracic aorta, in the peripheral vasculature, and in the brain, among many others and other lumen or false lumen filling applications. Any graft, stent-graft, balloon, or the like insertable into an aneurysm sac is suitable for use in the current disclosure as the insertable medical device, such as the ANEURX AAADVANTAGE®, TALENT®, and ENDURANT® stent-grafts manufactured by Medtronic, Inc. Such stent-grafts typically include a metallic scaffold supporting a synthetic material, such as a woven or unwoven mesh or fabric that is placed over, within or around the scaffold. The stent-graft expands into place after being delivered through an EVAR procedure, as is known in the art. Although the stent-graft shown in FIG. 1 is a so-called “branched” or “bifurcated” stent-graft because it branches into legs 151, 152, it should be recognized that unbranched stent-grafts (i.e., stent-grafts that are not bifurcated into legs) are suitable for use in the present disclosure. Also suitable for use in the present disclosure are fenestrated stent-grafts, as are known in the art. The present invention could also be used with temporary implants such as “kissing balloons” and the like which are removed after the aneurysm is filled with an in-situ solidifying prepolymer formulation as described herein. In such embodiments, one or more balloons can be used to form a temporary lumen. In these embodiments, injected prepolymer formulation flows around the balloon(s). After the formulation solidifies, the balloon(s) may be removed such that a lumen is present in the remaining solid material.

Regardless of whether a branched or unbranched stent-graft is used, the stent-graft will include a first end 160, second end 161 and/or 162, and a lumen 170 extending there between. The first end 160 of stent-graft 150 is secured to a first end 111 of aneurysm 110. As used herein, a graft or stent-graft is said to be “secured” to the end of an aneurysm if it is held into contact with surrounding tissue, such as by friction fit without the use of any securing means or alternatively with the use of such securing means such as sutures, adhesives, or other suitable securing means. The second end 161 and/or 162 of stent-graft 150 is secured to a second end 112 of aneurysm 110 to span the aneurysm and form a stable channel for blood flow within abdominal aorta 115.

As an alternative to stent-grafts, the present disclosure may be used with tubular grafts that are unsupported by stent scaffolds. As another alternative, the present disclosure may be used with one or more inflatable balloons, which are temporarily inserted into the patient as the medical device, around which the in-situ solidifying prepolymer formulation is delivered. As another alternative, the present disclosure may be used with another medical device (e.g., a stent, graft, stent graft, balloon, particles, embolization coil, flow diverter, flow disruptor, embolization plug, embolic filter, or other barrier).

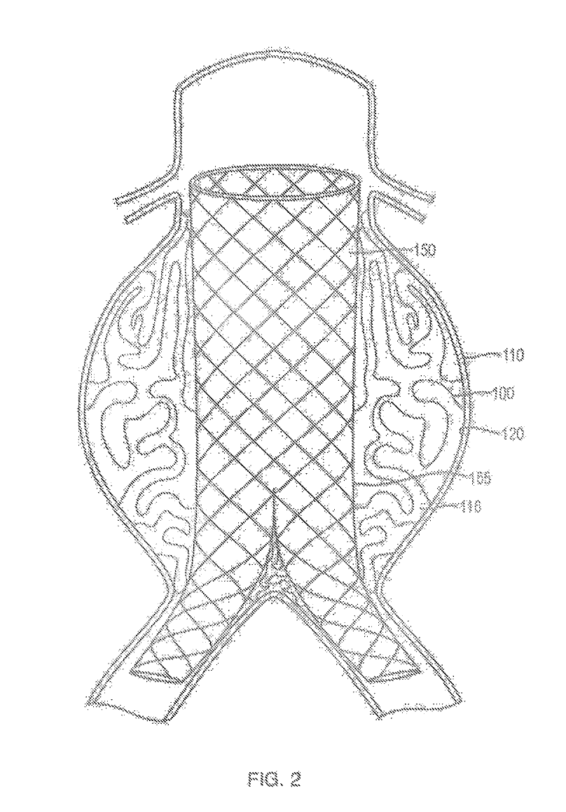

In accordance with an embodiment of the present disclosure, after the graft, stent-graft or balloon is placed within an aneurysm, an in-situ solidifying prepolymer formulation is inserted between an exterior surface 155 of the medical device (such as stent-graft 150) and a tissue surface 120 of aneurysm 110. In a preferred embodiment as shown in FIG. 2, the in-situ solidifying prepolymer formulation 100 may substantially fill the aneurysm sac 116. Because of the in-situ forming nature of the implant 100, it preferably flows to contact the graft and substantially all tissue surfaces defining the aneurysm sac 116, including penetrating to some degree into blood vessels and any other lumens opening into the aneurysm. Alternatively, the implant 100 may only partially fill the aneurysm sac 116. In various embodiments, the implant 100 is placed into contact with the exterior surface 155 of stent-graft 150, the tissue surface 120 of aneurysm 110, both of these surfaces, or neither of these surfaces. The exterior surface 155 of the medical devices of the present disclosure are preferably generally substantially solid, meaning that they include some porosity but are sufficiently solid to prevent substantial quantities of implant-forming formulation from flowing there-through.

The implant is formed in-situ substantially commensurately with the delivery of an implant-forming prepolymer formulation into the aneurysm sac, whereupon it reacts with water in the blood present within the sac, or with saline, water or other suitable fluid delivered together with the prepolymer formulation, or with another water-containing environment. Such fluid may pre-exist at the delivery site (as in the case of blood) in a so-called “one-part system,” or it may be delivered to the site concurrently with the prepolymer formulation or it may be pre-mixed with the prepolymer formulation shortly before delivery in so-called “two-part systems.” In such two-part systems, the fluid delivered with (or pre-mixed with) the prepolymer formulation is preferably saline.

The in-situ solidifying prepolymer formulations of the present disclosure are delivered to a body cavity site using any suitable delivery means. In one embodiment, the prepolymer that forms the implant is delivered to an aneurysm through a delivery catheter 200, as shown in FIG. 3. The catheter 200 is generally an elongated tube having an open distal end 210 and a lumen 220 extending along the length of the tube. When placed within the aneurysm sac 116, the prepolymer formulation is extruded from the distal end 210, whereupon it reacts in the presence of blood or other fluid to form an implant 100 in-situ. In certain embodiments where two formulations are simultaneously injected, the catheter 200 may be a dual lumen catheter so as the keep the formulations separate until delivery. In various embodiments, the prepolymer formulation immediately reacts forming a cured outer “skin” and a slower hardening interior that permits retention at the target site, even under high flow conditions, while maintaining material flow and/or flexibility to fill complex geometries.

In certain embodiments, the implant 100 is a polysiloxane implant. The implant 100 may be formed in-situ, for example, from a one-part formulation that includes an α-aminomethylalkoxysilane functionalized polysiloxane prepolymer, or the implant 100 may be formed in-situ, for example, from a two-part formulation in which the first part of the formulation includes a α-aminomethylalkoxysilane functionalized polysiloxane prepolymer and the second part of the formulation includes, for example, an aqueous fluid or a (meth)acrylate-functionalized small molecule or polymer (e.g., butane diol diacrylate or a polysiloxane diacrylate, among many other possibilities).

In some embodiments, the catheter 200 includes a one-way valve near the distal end to prevent blood from wicking into the catheter and causing premature reaction of prepolymer formulation therein. In some embodiments, the catheter 200 includes a pressure sensor on or near distal end 210 to indicate completion of implant delivery. Alternately, a pressure sensor is incorporated on or near the proximal end to measure pressure in the delivery lumen.

In the present disclosure, “acrylate” is a generic term referring to acrylic acid or a salt or ester acrylic acid. In the present disclosure, “methacrylate” is a generic term referring to methacrylic acid or a salt or ester methacrylic acid. In the present disclosure, “(meth)acrylate” is a generic term referring to an acrylate and/or a methacrylate.

The term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20 or fewer. In some embodiments, a straight chain or branched chain alkyl may have 20 or fewer carbon atoms in its backbone (e.g., C₁-C₂₀ for straight chain, C₃-C₂₀ for branched chain), 12 or fewer, 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclohexyl, and the like.

The terms “alkenyl” and “alkynyl” refer to unsaturated aliphatic groups analogous in length and possible substitution to the alkyls described above, but that contain at least one double or triple bond respectively.

The term “hydrocarbon” refers to groups consisting of hydrogen and carbon atoms and includes alkyl, alkenyl and alkynyl groups.

As used herein, the term “halogen” or “halide” designates —F, —Cl, —Br, or —I.

The term “alkoxy” refers to the group, —O-alkyl.

The terms “amine” and “amino” are art-recognized and refer to primary, secondary and tertiary amines, e.g., a moiety that can be represented by the general formula: N(R′)(R″)(R″) wherein R′, R″, and R′″ each independently represent a group permitted by the rules of valence, for example, hydrogen, an alkyl group, an alkenyl group or an alkynyl group, among many other possibilities.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocycyl, aromatic or heteroaruomatic moieties, —CF3, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like.

The present disclosure is further described with reference to the following non-limiting example.

### Example 1

### Synthesis of α-Aminomethyltriethoxysilane End-Terminated Polydimethylsiloxane

In a one liter three-neck round bottom flask equipped with a reflux condenser and gas adaptor, 394.17 grams of aminopropyl terminated polydimethylsiloxane (0.67 wt % amine) was added. The polymer was cycled under vacuum and argon three times and left under argon for the subsequent steps. Using syringes, 21.497 grams of triethylamine, 44.855 grams of chloromethyltriethoxysilane, and 200 mL of anhydrous toluene were added to the flask. The reaction temperature was then raised to 85° C. and mixed 20 hours. The flask was brought to room temperature and the triethylamine hydrochloride precipitate was filtered out using a medium porosity filter funnel under argon. The remaining volatiles were removed under high vacuum at 110° C. The final product remaining in the flask was α-aminomethyltriethoxysilane end-terminated polydimethylsiloxane (yield=395.11 grams; viscosity=440 cP at 22° C.).

Other α-aminomethylethoxysilane end-terminated polydimethylsiloxanes (AMES-PDMS) have been prepared using different aminopropyl modified polydimethylsiloxanes (AP-PDMS) and/or different chloromethylethoxysilanes (CMES), with results summarized in the following table:

| Pre- | AP-PDMS | | | | AMES-PDMS |
|---|---|---|---|---|---|
| polymer | Amine | Linear/ | Function- | | viscosity |
| # | wt % | Branched | ality | CMES | (cP @ 22° C.) |
| PP1 | 1.37% | Branched | 3 | CMTES | 3836 |
| PP2 | 0.67% | Linear | 2 | CMTES | 440 |
| PP3 | 3.53% | Linear | 2 | CMTES | 89 |
| PP4 | 1.37% | Branched | 3 | CMMDES | 963 |
| PP5 | 1.09% | Linear | 2 | CMTES | 363 |
| PP6 | 1.37% | Branched | 3 | CMMDES | 1533 |
| PP7 | 1.37% | Branched | 4 | CMMDES | 612 |

CMTES = chloromethyltriethoxysilane
CMMDES = chloromethylmethyldiethoxysilane

### Example 2

### Filling of Model Human Aortic Aneurysm

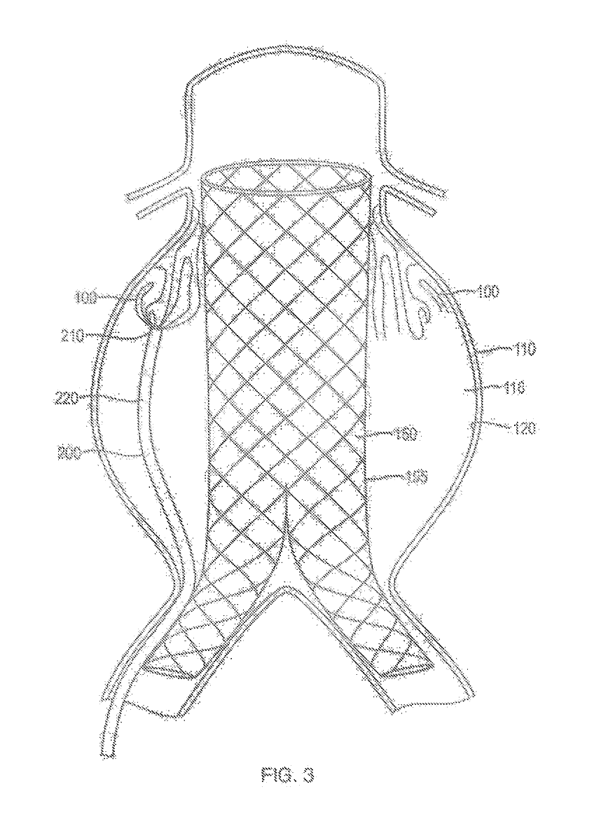

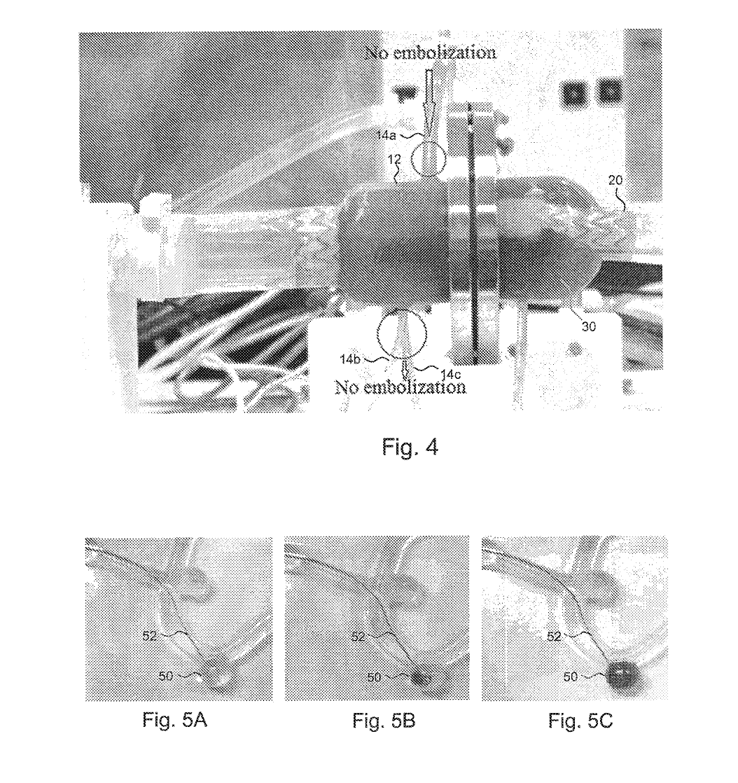

In this example, a one-part prepolymer formulation as described herein was found to be to be effective in filling a model of a human aortic aneurysm made from molded silicone as shown in FIG. 4. The aneurysm model was similar in size to a human abdominal aortic aneurysm, has a volume of approximately 120 mL and a complex geometry in which three silicone tubes 14a, 14b, 14c were attached within the aneurysmal segment to mimic the inferior mesenteric artery and two lumbar arteries. These tubes were in fluid communication with the aneurysm 12. A bifurcated sent graft 20, specifically a 36×20 mm stent graft with a 16 mm extension, was placed across the aneurysmal segment. The silicone aneurysm model was filled with water.

The formulation comprised an α-aminomethylalkoxysilane functionalized polydimethylsiloxane prepolymer as described herein with tantalum suspended as a visualization agent. The formulation is then delivered via an 8F catheter into the space between the graft and the aneurysm wall. It effectively filled 90+% of the space between the graft and the model aneurysm wall and corresponds to the dark area 30 within the aneurysm of FIG. 4. The formulation reacted quickly as described herein and immediately formed a thick “skin” upon contact with water. The liquid contained within the formulation “skin” allowed the material remain flexible and to flow around the stent graft without causing crushing or impingement of the stent graft. Moreover, no flow was observed outside the target area (i.e., out of the aneurysm segment into the three tubes that mimic the inferior mesenteric artery and two lumbar arteries), which is desirable as flow outside the target area could lead to embolism. Thus the present example, demonstrates the ability of moisture curing α-aminomethylalkoxysilane functionalized prepolymer formulations to fill large complex volumes such as those associated with aneurysms.

### Example 3

### Filling of Model Human Cerebral Aneurysm

In this example, a one-part prepolymer formulation as described herein was found to be to be effective in filling a model of a human cerebral aneurysm made from molded silicone. The aneurysm model was similar in size to a human cerebral aneurysm with a diameter of approximately 10 mm. The model was filled with saline and a pump was used to maintain a flow rate of 100 mL/min through the parent artery to approximate physiological flow conditions.

Referring now to FIGS. 5A-5C, a formulation comprising an α-aminomethylalkoxysilane functionalized polydimethylsiloxane prepolymer with tantalum suspended as a visualization agent was loaded into a syringe and deployed into the aneurysm 50 of the model using a 0.021″ microcatheter 52. FIG. 5A shows the system prior to injection in which the microcatheter 52 is placed in the unfilled aneurysm; FIG. 5B shows the system at a time when filling of the aneurysm is in progress; and FIG. 5C shows the system after the aneurysm has been filled. The formulation was easily deployed by hand to fill the aneurysm. The formulation filled 100% of the aneurysm space with a very small projection into the parent vessel. No breakaways or pieces of the formulation became dislodged during or after the deployment, with the final implant remaining as a single, intact material that made conformal contact with the aneurysm. This example demonstrates the usefulness of moisture curing α-aminomethylalkoxysilane functionalized prepolymer formulations as medical device to fill small aneurysms.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, formulation, composition, system, article, material, kit, and/or method described herein. In addition, any combination of two or more such formulations, compositions, features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims. “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. An injectable formulation comprising a prepolymer that comprises a polymer backbone and a plurality of α-aminomethylalkoxysilane groups, wherein the injectable formulation has a viscosity of less than 2000 cp.

2. The injectable formulation of claim 1, wherein the polymer backbone is selected from a branched polymer backbone and/or a linear polymer backbone.

3. The injectable formulation of claim 1, wherein the polymer backbone is selected from a polysiloxane backbone and a polyalkylene oxide backbone.

4. The injectable formulation of claim 1, wherein the α-aminomethylalkoxysilane groups are selected from α-aminomethylalkoxysilane side groups, α-aminomethylalkoxysilane terminal groups, and combinations of the same.

5. The injectable formulation of claim 1, wherein the α-aminomethylalkoxysilane groups are selected from α-aminomethyltrialkoxysilane groups, α-aminomethyldialkoxyalkylsilane groups and α-aminomethylalkoxydialkylsilane groups.

6. The injectable formulation of claim 1, wherein the α-aminomethylalkoxysilane groups are α-aminomethylethoxysilane groups.

7. The injectable formulation of claim 1, wherein the α-aminomethylalkoxysilane groups comprise a —NH—CH₂—SiOR¹ᵢR²₃₋ᵢ group, where R¹ is a C1-C10 hydrocarbon radical, R² is a C1-C10 hydrocarbon radical that may be the same as or different from R¹, and i=1, 2, or 3.

8. The injectable formulation of claim 7, wherein R¹ is —CH₂CH₃.

9. The injectable formulation of claim 1, wherein the α-aminomethylalkoxysilane groups comprise a —NH—CH₂Si(OCⱼH₂ⱼ₊₁)ᵢ(CₖH₂ₖ₊₁)₃₋ᵢ group, where j ranges from 1 to 10, k ranges from 1 to 10, and i=1, 2, or 3.

10. The injectable formulation of claim 1, wherein the α-aminomethylalkoxysilane groups comprise a -L-NH—CH₂—Si(OCⱼH₂ⱼ₊₁)(CₖH₂ₖ₊₁)₃₋ᵢ group, where j ranges from 1 to 10, k ranges from 1 to 10, i=1, 2, or 3, and L is selected from a C1-C20-hydrocarbon, a polysiloxane and a poly(alkylene oxide).

11. The injectable formulation of claim 9, wherein j=1, 2 or 3 and k=1, 2 or 3.

12. The injectable formulation of claim 9, wherein j=2.

13. The injectable formulation of claim 1, wherein the injectable formulation has a viscosity ranging from 10 cp to 1000 cp.

14. The injectable formulation of claim 1, wherein the injectable formulation has a viscosity ranging from 10 cp to 250 cp.

15. The injectable formulation of claim 1, further comprising a solvent.

16. The injectable formulation of claim 15, wherein the solvent is selected from ethanol, dimethylsulfoxide (DMSO), diglyme, polyethyleneoxide, polyethylene glycol dimethyl ether, propylene glycol, polypropylene glycol, acetone, dimethyl isosorbide, triacetin, N-methylpyrrolidone, and triethyl citrate.

17. The injectable formulation of claim 1, further comprising an imaging contrast agent.

18. The injectable formulation of claim 17, wherein the imaging contrast agent is selected from a radiopaque agent and/or an ultrasound contrast agent.

19. The injectable formulation of claim 1, disposed in the sealed container.

20. The injectable formulation of claim 1, disposed in the sealed container selected from a capped syringe, a metal foil pouch and a glass vial.
